(19) United States
(12) Patent Application Publication
(10) Pub. No.: US 2010/0068741 A1
(43) Pub. Date: Mar. 18, 2010

(54) ASSAY SYSTEM FOR ADENOSINE TRIPHOSPHATE AND CREATINE KINASE

(75) Inventors: Daniel Sobek, Portola Valley, CA (US); Jianghong Rao, Sunnyvale, CA (US)

Correspondence Address: LAW OFFICES OF MIKIO ISHIMARU, 333 W. EL CAMINO REAL, SUITE 330, SUNNYVALE, CA, 94087, US

(73) Assignee: ZYMERA, INC., Portola Valley, CA (US)

(21) Appl. No.: 12/445,384

(22) PCT Filed: Oct. 17, 2007
(86) PCT No.: PCT/US07/81702

§ 371 Date: Apr. 13, 2009

Related U.S. Application Data
(60) Provisional application No. 60/829,880, filed on Oct. 17, 2006.

(51) Int. Cl. C12Q 1/48

(52) U.S. Cl. 435/15

(57) ABSTRACT

An assay method includes providing a luminescent nanocrystal; combining a solution having an adenosine triphosphate molecule; and displaying a light emission by the luminescent nanocrystal and the solution combined.

### CROSS-REFERENCE TO RELATED APPLICATION(S)

This application claims the benefit of U.S. Provisional Patent Application Ser. No. 60/829,880 filed Oct 17, 2006.

### TECHNICAL FIELD

The present invention relates to an assay system for measuring adenosine triphosphate (ATP) and creatine kinase using Bioluminescence Resonance Energy Transfer (BRET).

### BACKGROUND ART

Adenosine triphosphate (ATP) is an energy source for biochemistry (FIG. 1). In anabolic reactions one phosphate group from ATP is transferred to a second molecule and in catabolic reactions one phosphate group is added to adenosine diphosphate (ADP) to produce ATP.

Adenosine diphosphate is an important intermediate in cellular metabolism as the partially dephosphorylated form of adenosine triphosphate. The compound is 5-adenylic acid with an additional phosphate group attached through a pyrophosphate bond. ADP is produced from adenosine triphosphate and reconverted to this compound in coupled reactions concerned with the energy metabolism of living systems.

The presence of ATP can be measured using firefly Luciferase bioluminescence. The bioluminescence created by this reaction can be correlated to the amount of ATP present in the sample. Bioluminescence ATP measurements may be applied to the detection of bacteria in food and food processing equipment, and to biodefense. The luciferin/luciferase bioluminescence reaction may also be employed as an indicator for ATP producing reactions such as the reverse reaction catalyzed by kinases (FIG. 2).

Creatine kinase (CK) is an enzyme found in skeletal muscle, brain, heart, and other organ tissues. Elevated creatine kinase levels in blood may signal diseases associated with skeletal, muscle, cardiac conditions, diseases of the central nervous system, or thyroid problems. At an optimal pH of 9, creatine kinase catalyzes phosphoryl transfer from adenosine triphosphate (ATP) into creatine, forming phosphocreatine and adenosine diphosphate (ADP). The reverse reaction is favored at a neutral pH with an optimum at a pH of 6.7. FIG. 3 illustrates the reactions catalyzed by this enzyme.

Most methods for measuring creatine kinase activity use the reverse reaction. In this case, CK catalyzes the phosphorylation of adenosine diphosphate (ADP) into adenosine triphosphate (ATP). An indicator reaction is usually employed to measure the amount of ATP produced in the first reaction. This indicator reaction may be implemented using firefly Luciferase bioluminescence.

One drawback of the firefly Luciferase indicator reaction is that it typically produces yellow and green emission that may be quenched by blood proteins such as hemoglobin. The assays made directly in body fluids such as whole blood may be difficult to read accurately due to hemoglobin absorption and self-fluorescence of the background from other blood proteins with natural fluorescence.

Some efforts have been made by using Bioluminescence Resonance Energy Transfer, a mechanism that allows the non-radiatively energy transfer from a bioluminescent enzyme directly into a fluorescent protein. The energy coupling is enabled by linking the bioluminescent donor and fluorescent protein in close proximity. This energy transfer enables shifting the emission of the bioluminescent reaction to the emission wavelength the fluorescent protein, which combination can be chosen to emit wavelengths in the green to yellow spectrum.

Thus, a need still remains for an assay system for ATP and CK based on bioluminescence resonance energy transfer sensing that can be implemented directly in whole blood or other body fluids. In view of the ever-increasing activity in the biosciences, it is increasingly critical that answers be found to these problems.

Solutions to these problems have been long sought but prior developments have not taught or suggested any solutions and, thus, solutions to these problems have long eluded those skilled in the art.

### DISCLOSURE OF THE INVENTION

The present invention provides an assay system including providing a luminescent nanocrystal, combining a solution having an adenosine triphosphate molecule, and displaying a light emission by the luminescent nanocrystal and the solution combined.

Certain embodiments of the invention have other aspects in addition to or in place of those mentioned above. The aspects will become apparent to those skilled in the art from a reading of the following detailed description when taken with reference to the accompanying drawings.

### BRIEF DESCRIPTION OF THE DRAWINGS

FIG. 1 is a bonding diagram of an adenosine triphosphate molecule;

FIG. 2 is a diagram of a generic reaction catalyzed by kinase enzymes;

FIG. 3 is a view of a reaction diagram catalyzed by creatine kinase;

FIGS. 4A and 4B are a view of a chemical reaction diagram for an assay system of adenosine triphosphate and creatine kinase, in an embodiment of the present invention;

FIG. 5 is a block diagram of a Bioluminescence Resonance Energy Transfer luminescent nanocrystal, in an embodiment of the present invention;

FIG. 6 is a block diagram of a light emission detection system, in an embodiment of the present invention; and

FIG. 7 is a flow chart of an assay system for adenosine triphosphate and creatine kinase in an embodiment of the present invention.

### BEST MODE FOR CARRYING OUT THE INVENTION

The following embodiments are described in sufficient detail to enable those skilled in the art to make and use the invention. It is to be understood that other embodiments would be evident based on the present disclosure, and that process or mechanical changes may be made without departing from the scope of the present invention.

In the following description, numerous specific details are given to provide a thorough understanding of the invention. However, it will be apparent that the invention may be practiced without these specific details. Likewise, the drawings showing embodiments of the system are semi-diagrammatic and not to scale and, particularly, some of the dimensions are for the clarity of presentation and are shown greatly exaggerated in the drawing FIGS. Where multiple embodiments are disclosed and described, having some features in common, for clarity and ease of illustration, description, and comprehension thereof, similar and like features one to another will ordinarily be described with like reference numerals.

The term “on” means there is direct contact among elements. The term “system” as used herein means and refers to the method and to the apparatus of the present invention in accordance with the context in which the term is used.

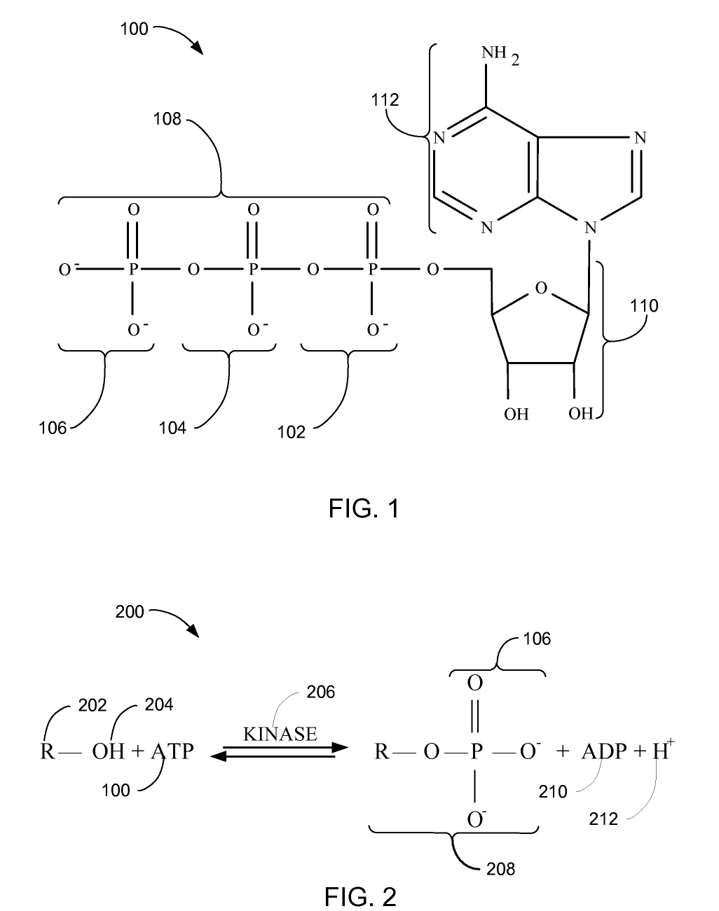

Referring now to FIG. 1, therein is shown a bonding diagram of an adenosine triphosphate (ATP) molecule 100. The bonding diagram of the adenosine triphosphate molecule 100 depicts a first phosphate group 102 coupled to a second phosphate group 104 coupled to a third phosphate group 106. A triphosphate chain 108 is formed of the first phosphate group 102, the second phosphate group 104, and the third phosphate group 106.

The triphosphate chain 108 is connected to a ribose molecule 110 which is then connected to an adenine molecule 112.

The adenosine triphosphate (ATP) molecule 100 may be used as an indicator for detecting a kinase. The adenosine triphosphate (ATP) molecule 100 is detectable by an embodiment of the present invention.

Referring now to FIG. 2, therein is shown a diagram of a generic reaction catalyzed by kinase enzymes 200. The diagram of the reaction depicts a chemical substrate (R) 202 containing a hydroxyl group 204 that may be in solution with the adenosine triphosphate (ATP) molecule 100 and is operated upon by a kinase 206, such as a creatine kinase, to yield products consisting of a phosphorylated product 208 containing the original substrate 202 and a phosphate group 106, an adenosine diphosphate (ADP) molecule 210, and a proton 212. In the generic reaction catalyzed by kinase enzymes 200 the kinase 206 catalyzes the transfer of a phosphate group 106 from the adenosine triphosphate (ATP) molecule 100 to the chemical substrate (R) 202, forming the phosphorylated product 208 consisting of the substrate (R) 202 linked to the phosphate group 106, and an adenoside diphosphate (ADP) 210 molecule.

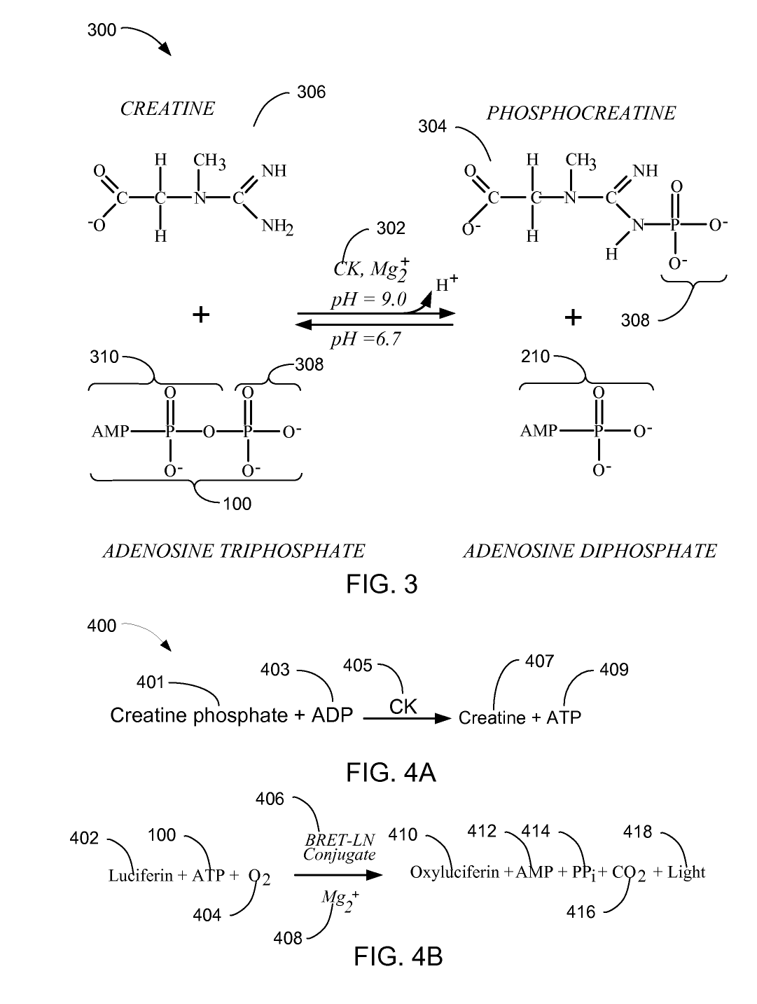

Referring now to FIG. 3, therein is shown diagram of the reaction catalyzed by creatine kinase 302. Similar to the generic kinase reaction in FIG. 2, creatine kinase (CK) 302, in the presence of magnesium (Mg₂), catalyzes the phosphorylation of creatine 300 into creatine phosphate 304, using a phosphate group 308 from ATP 100, leaving adenosine diphosphate (ADP) as a product 210. The phosphorylation of creatine is favored at a basic pH of 9.0 and the reverse reaction is favored at a pH of 6.7.

The reverse reaction may take place in the muscle tissue of a person working out. As the muscle consumes energy, the creatine kinase 302 may cleave the phosphate group 308 from the phosphocreatine molecule 304. When the phosphate group 308 is freed, it becomes bonded to the adenosine diphosphate (ADP) molecule 210 forming the adenosine triphosphate (ATP) molecule 100.

Referring now to FIG. 4A and 4B, therein are shown a chemical reaction diagram for an assay system of adenosine triphosphate and creatine kinase 400. The reaction diagram of the assay system for adenosine triphosphate and creatine kinase 400 depicts a creatine phosphate molecule 401 in solution with an adenosine diphosphate (ADP) molecule 403 catalyzed by a creatine kinase molecule 405. The reaction produces a creatine molecule 407 and an adenosine triphosphate molecule 409. In the reaction, the creatine kinase 405 may be contained in a sample of whole blood and is the limiting factor in determining how much the adenosine triphosphate molecule 409 is produced. In the second reaction the ATP will limit the reaction and determine the amount of the light emission 418 that will be activated.

Referring now to 4B, therein are shown a chemical reaction diagram for an assay system of adenosine triphosphate and creatine kinase 400. The reaction diagram of the assay system for adenosine triphosphate and creatine kinase 400 depicts a luciferin molecule 402 in solution with the adenosine triphosphate (ATP) molecule 100 and an oxygen (02) molecule 404. A Bioluminescence Resonance Energy Transfer luminescent nanocrystal (BRET-LN) conjugate 406 in the presence of magnesium (Mg₂) 408, catalyzes the reaction creating an oxyluciferin molecule 410, an adenosine monophosphate (AMP) molecule 412, a phosphate (PP) molecule 414, a carbon dioxide (CO₂) molecule 416, and a light emission 418. The assay system for adenosine triphosphate and creatine kinase 400 may provide an indicator reaction for detecting the amount of the adenosine triphosphate (ATP) molecule 100 produced by the previous reaction. In this case the amount of the adenosine triphosphate (ATP) molecule 100 is indicative of the catalytic activity of the creatine kinase (CK) 302 that catalyzed the production of the adenosine triphosphate (ATP) molecule 100 in the reaction of FIG. 3. The amount of the light emission 418 is directly related to the amount of the adenosine triphosphate (ATP) molecule 100 present in the solution.

The characteristic wavelength of the light emission 418 is dependent on the Bioluminescence Resonance Energy Transfer luminescent nanocrystal (BRET-LN) conjugate 406. The BRET-LN conjugate 406 may be designed to emit energy having a wavelength in the range of 600 nm to 900 nm. This range of the wavelength of the BRET-LN conjugate 406 is distinct from quenching or the possible self-fluorescence of the blood proteins found in whole blood or serum. The possible self-fluorescence of the blood proteins would occur in the wavelength range of 300 nm to 450 nm, and hemoglobin absorption is more prevalent at wavelengths up to 600 nm. With such a clear separation in the ranges of possible emissions from a serum, it is possible to filter the response from the blood proteins and leave only the light emission 418 from the detection of the adenosine triphosphate (ATP) molecule 100.

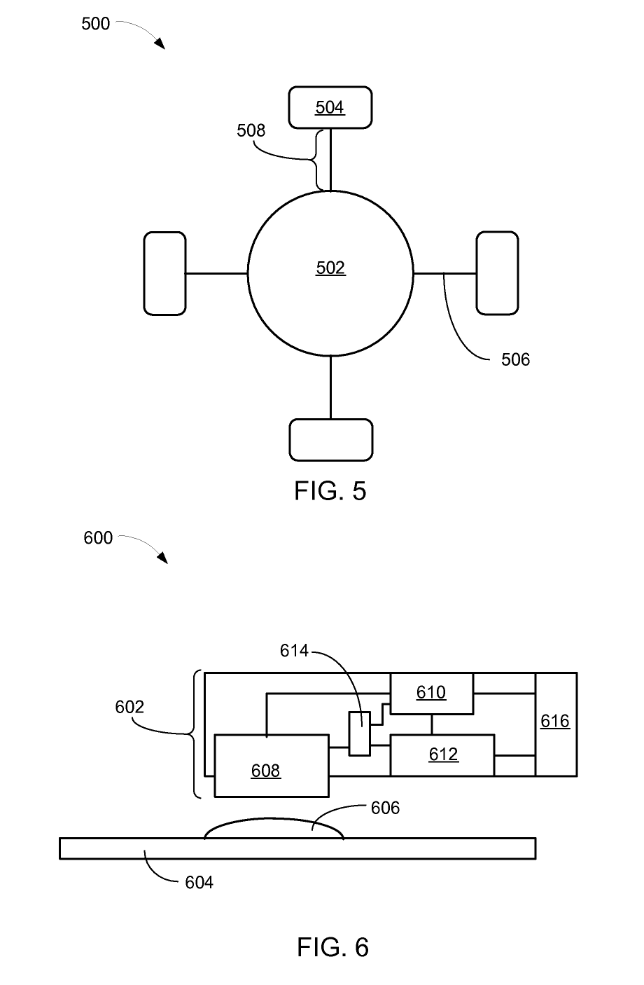

Referring now to FIG. 5, therein is shown a block diagram of a Bioluminescence Resonance Energy Transfer luminescent nanocrystal 500, in an embodiment of the present invention. The block diagram of the Bioluminescence Resonance Energy Transfer luminescent nanocrystal 500 depicts a semiconductor nanostructure 502, such as a bioluminescence resonance energy transfer acceptor molecule, linked to a luminescent enzyme 504, such as a bioluminescent enzyme or a chemiluminescent enzyme. The luminescent enzyme 504 may be held in position by a spacing molecule 506. The luminescent enzyme 504 must be held within a Foster distance 508, usually between 10 and 100 Angstroms, in order to allow Bioluminescence Resonant Energy Transfer to take place.

In an example of Bioluminescence Resonance Energy Transfer luminescent nanocrystal 500, the semiconductor nanostructure 502 may be linked, at the Foster distance 508 of 30 Angstroms, to the luminescent enzyme 504, such as a firefly luciferase, with maximum emission at a wavelength of 550 nm to 560 nm. When the Bioluminescence Resonance Energy Transfer luminescent nanocrystal 500 is activated, the luminescent enzyme 504 will activate the semiconductor nanostructure 502 through the Bioluminescent Resonance Energy Transfer. The semiconductor nanostructure 502 may be formulated to provide the light emission 418, of FIG. 4, at a wavelength of 600 nm to 900 nm

In the previous example, the Bioluminescent Resonance Energy Transfer luminescent nanocrystal (BRET-LN) conjugates 500 such as the semiconductor nanostructure 502 closely linked to the luminescent enzyme 504 that employs the adenosine triphosphate (ATP) molecule 100 as a co-substrate, such as firefly luciferase. The emission spectra and stability of firefly luciferase may be optimized through specific mutations. In the preferred implementation of the invention, the BRET-LN conjugate 500 would incorporate a mutant form of the luminescent enzyme 504 optimized for an efficient blue-shifted emission for better coupling to the luminescent nanocrystal and maximum stability.

In a preferred embodiment of the invention the semiconductor nanostructure 502 that may emit in the red visible light spectrum will be used as a BRET acceptor molecule.

Emissions at wavelengths longer than 650 nm minimize the possibility of light quenching from blood proteins such as hemoglobin.

There are many ways to achieve a stable linkage between the semiconductor nanostructure 502 and the luminescent enzyme 504. One method is to form a stable amide linkage between the two molecules using 1-ethyl-3-(3-dimethylaminopropyl)carbodiimide hydrochloride (EDC) as a coupling reagent. A second method that has the potential to better retain the activity of the luminescent enzyme 504 is to add a histadine tag to the luminescent enzyme 504, and conjugate nickel-nitrilotriacetate (NTA) to the semiconductor nanostructure 502 in the presence of nickel ions. A third method involves using a streptavidin-biotin bond, with streptavidin on the surface of the semiconductor nanostructure 502 and biotin-conjugated with the luminescent enzyme 504. There are many other methods that could be employed to create the BRET-LN conjugate 500 incorporating firefly luciferase.

Referring now to FIG. 6, therein is shown a block diagram of a light emission detector system 600, in an embodiment of the present invention. The block diagram of the light emission detector system 600 depicts an emission detection device 602 positioned over a platform 604 having a solution 606, such as whole blood, plasma, or serum catalyzed by creatine kinase 302 of FIG. 3, containing the luminescent nanocrystal 500 in the solution.

The emission detection device 602 may include an emission sensor 608 coupled to a memory device 610. A processor 612 may be used to manipulate the data stored in the memory device 610. A power source 614 may be coupled to the emission sensor 608, the memory device 610, and the processor 612. An interface device 616 may be coupled to the memory device 610 and the processor 612 for transfer or display of the data detected by the emission sensor 608.

The embodiment of the emission detection device 602 is an example only and is not intended to limit the implementation of the present invention. The emission detection device 602 may be a hand held instrument having the power source 614 such as a battery, or it may be part of a larger instrument having the power source 614 located differently or using some other type of power.

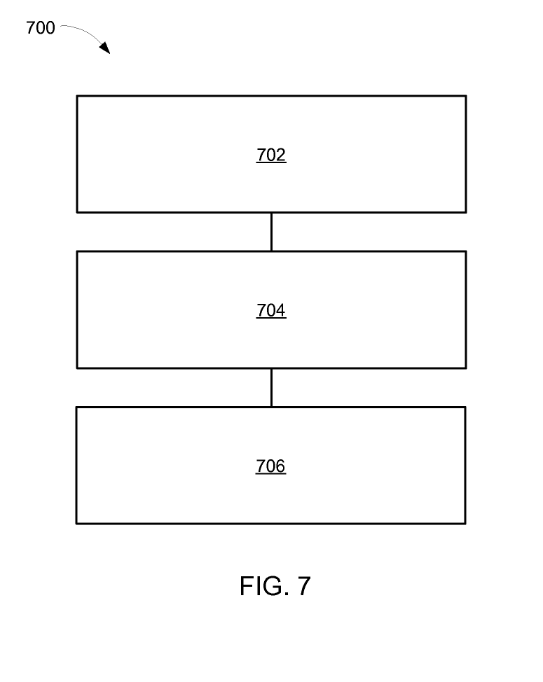

Referring now to FIG. 7, therein is shown a flow chart of an assay system for adenosine triphosphate and creatine kinase 700, for operating the assay system for adenosine triphosphate and creatine kinase 400, in an embodiment of the present invention. The system 700 includes providing a luminescent nanocrystal in a block 702; combining a solution having an adenosine triphosphate molecule in a block 704; and displaying a light emission by the luminescent nanocrystal and the solution combined in a block 706.

It has been unexpectedly discovered that an assay system may be readily fabricated to detect adenosine triphosphate (ATP) and creatine kinase (CK) in a blood sample utilizing a luminescent nanocrystal.

It has been discovered that the present invention thus has numerous aspects.

A principle aspect that has been unexpectedly discovered is that the present invention provides an accurate indicator of the presence of ATP and CK in a blood sample.

Another aspect is that this assay is reliable and transportable, making it a benefit for patients and practitioners.

Yet another important aspect of the present invention is that it valuably supports and services the historical trend of reducing costs, simplifying systems, and increasing performance.

These and other valuable aspects of the present invention consequently further the state of the technology to at least the next level.

Thus, it has been discovered that the assay system for adenosine triphosphate and creatine kinase of the present invention furnishes important and heretofore unknown and unavailable solutions, capabilities, and functional aspects for enzyme analysis in blood. The resulting processes and configurations are straightforward, cost-effective, uncomplicated, highly versatile and effective, can be surprisingly and unobviously implemented by adapting known technologies, and are thus readily suited for efficiently and economically manufacturing blood analysis devices fully compatible with conventional manufacturing processes and technologies. The resulting processes and configurations are straightforward, cost-effective, uncomplicated, highly versatile, accurate, sensitive, and effective, and can be implemented by adapting known components for ready, efficient, and economical manufacturing, application, and utilization.

While the invention has been described in conjunction with a specific best mode, it is to be understood that many alternatives, modifications, and variations will be apparent to those skilled in the art in light of the aforegoing description. Accordingly, it is intended to embrace all such alternatives, modifications, and variations that fall within the scope of the included claims. All matters hithertofore set forth herein or shown in the accompanying drawings are to be interpreted in an illustrative and non-limiting sense.

1. An assay method comprising:
providing a luminescent nanocrystal;
combining a solution having an adenosine triphosphate molecule; and displaying a light emission by the luminescent nanocrystal and the solution combined.

2. The method as claimed in claim 1 wherein providing the luminescent nanocrystal includes providing a semiconductor nanostructure.

3. The method as claimed in claim 1 wherein providing the luminescent nanocrystal includes linking a luminescent enzyme.

4. The method as claimed in claim 1 wherein displaying the light emission includes detecting the adenosine triphosphate molecule in the solution.

5. The method as claimed in claim 1 further comprising fabricating an emission detection device for detecting the light emission from the luminescent nanocrystal.

6. An assay system comprising:
a luminescent nanocrystal;
a solution having an adenosine triphosphate molecule; and
a light emission by the luminescent nanocrystal and the solution combined.

7. The system as claimed in claim 6 wherein the luminescent nanocrystal includes a semiconductor nanostructure.

8. The system as claimed in claim 6 wherein the luminescent nanocrystal includes a luminescent enzyme linked.

9. The system as claimed in claim 6 wherein the light emission includes the adenosine triphosphate molecule detected in the solution.

10. The system as claimed in claim 6 further comprising an emission detection device for detecting the light emission from the luminescent nanocrystal.

11. The system as claimed in claim 6 further comprising:
a creatine kinase catalyzed the solution; and
the light emission includes the adenosine triphosphate molecules in the solution measured.

12. The system as claimed in claim 11 wherein the luminescent nanocrystal includes a semiconductor nanostructure for tuning a bioluminescent resonance energy transfer acceptor to emit the light emission having a wavelength in the range of 600 nm to 900 nm.

13. The system as claimed in claim 11 wherein the luminescent nanocrystal includes a luminescent enzyme by a bioluminescent enzyme or a chemiluminescent enzyme linked.

14. The system as claimed in claim 11 wherein the light emission includes the adenosine triphosphate molecule detected in the solution without a self-fluorescence in the solution.

15. The system as claimed in claim 11 further comprising an emission detection device that detects the light emission from the luminescent nanocrystal and provides an interface to display the light emission detected.

16. An assay method comprising:
providing a luminescent nanocrystal for indicating an adenosine triphosphate molecules detected;
combining a solution having the adenosine triphosphate molecule including catalyzing by a creatine kinase; and
displaying a light emission by the luminescent nanocrystal and the solution combined in which displaying the light emission includes measuring the adenosine triphosphate molecules in the solution.

17. The method as claimed in claim 16 wherein providing the luminescent nanocrystal includes providing a semiconductor nanostructure including tuning a bioluminescent resonance energy transfer acceptor for emitting the light emission having a wavelength in the range of 600 nm to 900 nm.

18. The method as claimed in claim 16 wherein providing the luminescent nanocrystal includes linking a luminescent enzyme including linking a bioluminescent enzyme or a chemiluminescent enzyme.

19. The method as claimed in claim 16 wherein displaying the light emission includes detecting the adenosine triphosphate molecule in the solution including preventing a self-fluorescence in the solution.

20. The method as claimed in claim 16 further comprising fabricating an emission detection device for detecting the light emission from the luminescent nanocrystal including providing an interface for displaying the light emission detected.
